(19) United States
(12) Patent Application Publication
(10) Pub. No.: US 2009/0225275 A1
(43) Pub. Date: Sep. 10, 2009

(54) VISION RECOVERY TRAINING APPARATUS

(75) Inventors: Hidenori Horie, Kanagawa (JP); Hisashi Akiyama, Kanagawa (JP); Eiichi Ookawa, Kanagawa (JP)

Correspondence Address: PEARNE & GORDON LLP, 1801 EAST 9TH STREET, SUITE 1200, CLEVELAND, OH, 44114-3108, US

(73) Assignees: MATSUSHITA ELECTRIC INDUSTRIAL CO., LTD., Osaka (JP); TECHNOMASTER CO., LTD., Kanagawa (JP)

(21) Appl. No.: 11/994,537

(22) PCT Filed: Jul. 5, 2006
(86) PCT No.: PCT/JP2006/313421

§ 371 Date: Jan. 29, 2008

(30) Foreign Application Priority Data
Jul. 5, 2005 (JP) 2005-196136
Sep. 27, 2005 (JP) 2005-280054
Jun. 30, 2006 (JP) 2006-180964

(51) Int. Cl. A61H 5/00; A61B 3/11

(52) U.S. Cl. 351/203; 601/37

(57) ABSTRACT

An object of the invention is to provide a vision recovery training apparatus capable of efficiently conducting vision recovery training and conducting training without inputting a training parameter according to an improvement state of an image formation adjustment function of each trainee every training.

In a vision recovery training apparatus 1 for training an image formation adjustment function of an eye, the vision recovery training apparatus of the invention comprises a display part 4 for displaying an image for vision recovery, and a main controller 11 which calculates a new training parameter based on a training parameter which is acquired from a storage device for storing a training history and is applied to a trainee at least one time and conducting training to which the calculated new training parameter is applied.

### TECHNICAL FIELD

The present invention relates to a vision recovery training apparatus for training an image formation adjustment function of an eye.

### BACKGROUND ART

Generally, a decrease in vision probably contributes to a decrease in an image formation adjustment function of an eye due to weakness or adjustment tension in an adjustment muscle (ciliary muscle) of the eye, and recovery of the vision can be expected by training this muscle. A method for improving vision by activating an accommodation function by activating this ciliary muscle by training has been performed conventionally.

For example, a vision recovery training apparatus for attempting to increase a vision improvement effect by activating action of the adjustment muscle described above has been proposed (for example, see Patent Reference 1). This vision recovery training apparatus is a device for moving a gaze plate in which proper characters or marks are written at proper speed, and a trainee conducts training of a ciliary muscle by gazing at the characters or marks of its gaze plate.

Also, a vision recovery training apparatus comprising an eyepiece part, a target capable of displaying a proper graphic form capable of being visually checked from the eyepiece part, a target movement device which moves the target between two predetermined distances from the eyepiece part at proper speed, and a display controller for changing a size of a displayed graphic form according to a distance between the eyepiece part and the target has been proposed (for example, see Patent Reference 2). Since this vision recovery training apparatus changes a display size of an image for vision training in proportion to an optical distance between a position of an eye of a trainee and a position of a display part for displaying the image for vision training, the display size of the image for vision training is changed so as to project the image for vision training on a retina of the trainee in a constant size according to the optical distance between the position of the eye of the trainee and the position of the display part for displaying the image for vision training. According to this configuration, a user of this apparatus gazes at a graphic form through the eyepiece part and thereby a focal point of the eye continuously moves between a short distance and a long distance and further a size of the graphic form in which an image is formed on a retina becomes constant, so that an adjustment muscle is adjusted efficiently.

In the vision recovery training apparatus described above, training information as to whether both eyes are trained at once or either eye is trained, display information about shape, color, etc. of a graphic form displayed, distance information about a near point, a far point, etc., a target movement speed profile between a far point and a near point, training time, etc. are variable. This is consideration capable of finely combining parameters of vision recovery so that the best training result can be achieved according to the extent, causes, etc. of a decrease in vision of a trainee.

Patent Reference 1: JP-A-6-339501

Patent Reference 2: International Publication Pamphlet 2004/066900

### DISCLOSURE OF THE INVENTION

### Problems that the Invention is to Solve

However, in the conventional vision recovery training apparatus as described above, even when an image formation adjustment function is improved by vision recovery training, a parameter (herein after called a training parameter) of vision recovery is fixed and it is necessary to again input the parameter if the training parameter attempts to be updated according to the extent of improvement in the image formation adjustment function. Particularly, when a trainee who is an object person of training is a young child, assistance of a training instructor may be required in order to input the training parameter. As a result of this, there were cases where efficiency of vision recovery training does not increase too much.

The invention has been implemented in view of the circumstances described above, and an object of the invention is to provide a vision recovery training apparatus capable of efficiently conducting vision recovery training and conducting training without inputting a training parameter according to an improvement state of an image formation adjustment function of each trainee every training.

### Means for Solving the Problems

A vision recovery training apparatus of the invention is a vision recovery training apparatus for training an image formation adjustment function of an eye, and comprises a training parameter calculator which calculates a new training parameter based on a training parameter which is acquired from a storage device which stores a training history and is applied to a trainee at least one time, and a controller for conducting training to which the calculated new training parameter is applied.

According to the configuration described above, vision recovery training can be conducted efficiently without inputting a training parameter every training since training according to an improvement state of an image formation adjustment function can be conducted for each trainee by conducting training to which a new training parameter based on a training parameter applied in the past is applied.

Also, the vision recovery training apparatus of the invention comprises a display device which displays an image for vision recovery training, and the controller performs control of the display device based on the new training parameter.

According to the configuration described above, vision recovery training can be conducted efficiently without inputting a training parameter every training since training according to an improvement state of an image formation adjustment function can be conducted for each trainee by performing control of the display device based on the new training parameter.

Also, in the vision recovery training apparatus of the invention, the controller performs display control of the display device based on the new training parameter.

According to the configuration described above, vision recovery training can be conducted efficiently without inputting a training parameter every training since training according to an improvement state of an image formation adjustment function can be conducted for each trainee by performing display control of the display device based on the new training parameter.

Also, in the vision recovery training apparatus of the invention, the controller changes a display size of the image for vision training so as to project the image for vision training on a retina of the trainee in a constant size according to an optical distance between a position of an eye of the trainee and a position of the display device.

According to the configuration described above, an adjustment muscle can be trained efficiently since a size of a graphic form imaged on a retina becomes constant.

Also, in the vision recovery training apparatus of the invention, the controller performs movement control of the display device based on the new training parameter.

According to the configuration described above, vision recovery training can be conducted efficiently without inputting a training parameter every training since training according to an improvement state of an image formation adjustment function can be conducted for each trainee by performing movement control of the display device based on the new training parameter.

Also, in the vision recovery training apparatus of the invention, the storage device stores the number of past trainings of a trainee as the training history, and the training parameter calculator calculates the new training parameter based on the number of the past trainings.

According to the configuration described above, a training parameter can be changed according to the number of past trainings of a trainee.

Also, in the vision recovery training apparatus of the invention, the storage device stores training conduct time as the training history, and the training parameter calculator calculates the new training parameter based on the training conduct time.

According to the configuration described above, frequency of the past training of a trainee can be discerned and a training parameter can be changed according to the frequency of the past training.

Also, in the vision recovery training apparatus of the invention, the storage device stores the new training parameter.

According to the configuration described above, a new training parameter according to the newest training state of a trainee can be calculated.

Also, the vision recovery training apparatus of the invention comprises a diameter measurement device which measures a diameter of a pupil of a trainee, and the training parameter calculator refers to correspondence between a training parameter and a change in a diameter of a pupil measured and calculates a new training parameter. Also, the vision recovery training apparatus of the invention comprises an imaging device which captures an image of a pupil of a trainee, and the diameter measurement device measures a diameter of a pupil of the trainee from an image captured by the imaging device.

While it was conventionally necessary to measure a training effect after training was conducted in order to grasp the training effect, according to the configuration described above, the training effect can be grasped during the training. Further, training can be conducted using a new training parameter according to a training effect properly without interrupting the training, so that efficient training can be conducted.

Also, the vision recovery training apparatus of the invention comprises an acquisition device which acquires biological information about a trainee, and a retrieval device which refers to a storage device which stores biological information about a trainee and retrieves biological information matched with the biological information acquired by the acquisition device.

According to the configuration described above, a trainee can be pinpointed easily using biological information without requesting an input of identification information etc. unique to the trainee from the trainee, so that the most suitable training parameter can be applied to each trainee without operating a parameter input etc. Consequently, training can be conducted efficiently by the training parameter suitable for each trainee.

Also, the vision recovery training apparatus of the invention comprises a selector which selects a training parameter corresponding to the biological information retrieved by the retrieval device.

According to the configuration described above, the most suitable training parameter can be applied to each trainee without performing an input operation of a parameter.

Also, the vision recovery training apparatus of the invention comprises a controller in which when biological information matched with the biological information acquired by the acquisition device is not present as a result of retrieval by the retrieval device, an input of a training parameter is prompted and the inputted training parameter and the biological information acquired by the acquisition device are associated and are stored in the storage device.

According to the configuration described above, a training parameter and biological information can be associated and stored for a new trainee.

Also, in the vision recovery training apparatus of the invention, the biological information can be acquired in a state of conducting training. Also, in the vision recovery training apparatus of the invention, the biological information is an iris of an eyeball.

According to the configuration described above, a trainee can input biological information without consciousness, so that convenience can be improved.

Also, in the vision recovery training apparatus of the invention, the acquisition device acquires biological information by a camera having sensitivity outside a wavelength of visible light.

According to the configuration described above, even under atmosphere of the absence of visible light, a shape of an iris present in an eyeball of a trainee can be captured and biological information can be acquired.

Also, the vision recovery training apparatus of the invention comprises the storage device.

Also, in the vision recovery training apparatus of the invention, the storage device is connected through a network.

According to the configuration described above, it becomes unnecessary to dispose the storage device in the vision recovery training apparatus itself, so that a configuration of the apparatus can be simplified and also a training parameter can be shared between plural vision recovery training apparatus connected to a network.

### ADVANTAGE OF THE INVENTION

According to the invention, vision recovery training can be conducted efficiently without inputting a training parameter every training since training according to an improvement state of an image formation adjustment function can be conducted for each trainee by conducting training to which a new training parameter based on a training parameter applied in the past is applied.

### BRIEF DESCRIPTION OF THE DRAWINGS

FIG. 1 is a diagram showing a configuration of a vision recovery training apparatus in a first embodiment of the invention.

FIG. 2 is a block diagram showing a configuration example of a main controller in the first embodiment of the invention.

FIG. 3 is a flow diagram showing a calculation example of training parameters in the first embodiment of the invention.

FIG. 4 is a diagram showing one example of a data format stored in a storage device in the first embodiment of the invention.

FIG. 5 is a diagram showing a configuration of a vision recovery training apparatus in a second embodiment of the invention.

FIG. 6 is a diagram specifically showing a configuration example of a main controller in the second embodiment of the invention.

FIG. 7 is a diagram showing a change in a pupil diameter of a trainee in the second embodiment of the invention.

FIG. 8 is a schematic diagram of an image formation state of a myopic person in the second embodiment of the invention.

FIG. 9 is a diagram showing the amount of change in a pupil diameter with respect to a training date in the second embodiment of the invention.

FIG. 10 is a block diagram showing a configuration of a vision recovery training apparatus of a third embodiment of the invention.

FIG. 11 is a block diagram specifically showing one example of a main controller of the vision recovery training apparatus of the third embodiment of the invention.

FIG. 12 is a diagram showing one example of a data format stored in a storage device in the third embodiment of the invention.

### DESCRIPTION OF REFERENCE NUMERALS AND SIGNS

1,100,300 VISION RECOVERY TRAINING APPARATUS
2,340 TRAINEE
3 CABINET
4,312 DISPLAY PART
5,303 EYEPIECE PART
6,314 SHIELDING PART
7,304 TRANSPORT PART
8,9,305,306 PULLEY
10,311 MOTOR
11,111,318 MAIN CONTROLLER
12,316 INPUT DEVICE
13,317 OPERATION DISPLAY PART
20,120 CENTRAL PROCESSING PART
21 DISPLAY CONTROL PART
22 MOTOR DRIVING CONTROL PART
23 OPENING AND CLOSING CONTROL PART
24 OPERATION INPUT PART
25 OPERATION DISPLAY CONTROL PART
26,126 STORAGE DEVICE
101,313 CAMERA
150 PUPIL DIAMETER CURVE
200 VISUAL TARGET
201 PUPIL
202 LENS (CRYSTALLINE LENS)
203 FOCAL DISTANCE
204 IMAGE FORMATION POINT
205 RETINA
206 VISUAL TARGET IMAGE
207 PUPIL DIAMETER
250 AMOUNT OF CHANGE IN PUPIL DIAMETER

### BEST MODE FOR CARRYING OUT THE INVENTION

A vision recovery training apparatus of embodiments of the invention will herein after be described using the drawings.

### First Embodiment

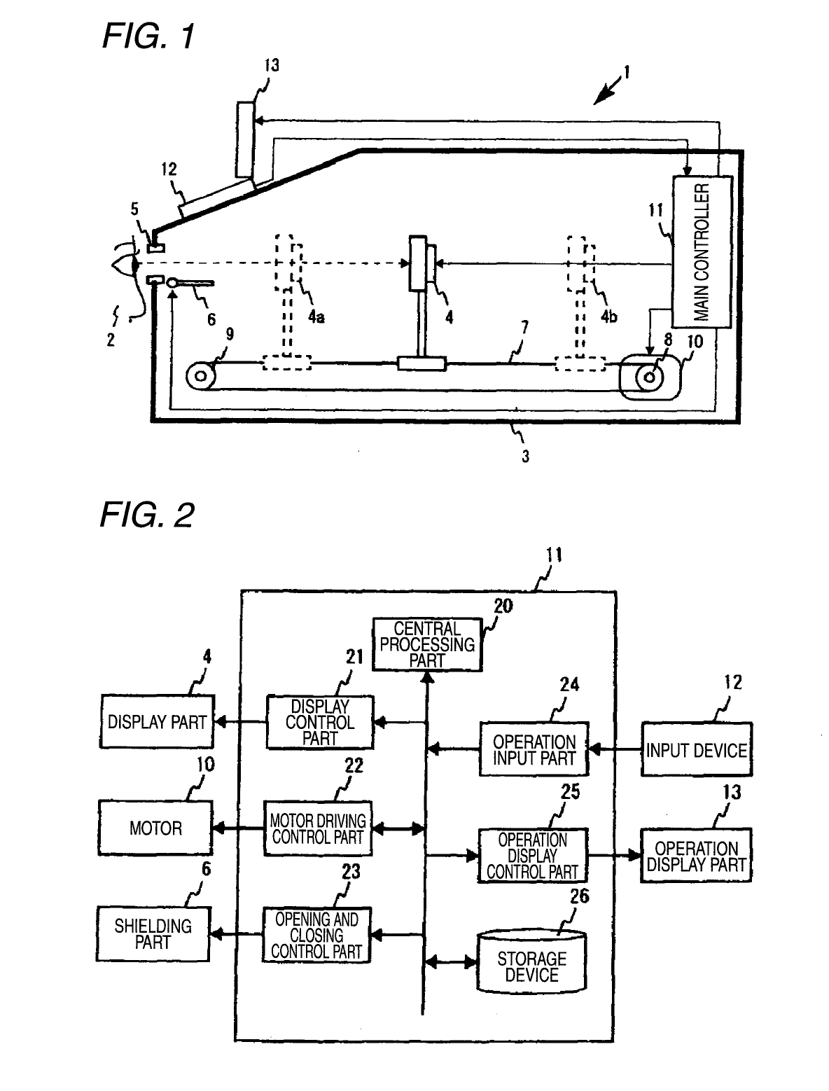

FIG. 1 is a diagram showing a configuration of a vision recovery training apparatus in a first embodiment of the invention. A vision recovery training apparatus 1 is an apparatus for training an image formation adjustment function of an eye of a trainee 2, and a display part 4 for displaying an image for vision training is arranged inside a cabinet 3. The diagram shows a state in which the display part 4 is positioned in the middle of a near point 4a and a far point 4b.

The trainee 2 using the present apparatus 1 puts the eyes to an eyepiece part 5, and looks at a character or a graphic form displayed on the display part 4, and conducts vision improvement training. It is desirable that the eyepiece part 5 have a size in which the trainee 2 can peep with both eyes, but one eyepiece part having a size of the extent capable of peeping with one eye may be prepared to conduct vision improvement training alternately by one eye. Also, two eyepiece parts having a size of the extent capable of peeping with one eye may be prepared to simultaneously conduct vision improvement training of both eyes, or one eyepiece part 5 may be blocked by an openable and closable shielding part 6 to conduct training of one eye or to alternately conduct training of right and left eyes. In addition, by disposing a lens in the eyepiece part 5 and forming a virtual image, the depth can also be decreased to miniaturize the apparatus.

Also, the display part 4 is constructed by an electrical display device such as an organic EL (Electro-Luminescence) display, a CRT (Cathode Ray Tube) display or a liquid crystal display. Background colors, colors, sizes, kinds, etc. of characters or graphic forms displayed by this display part 4 can easily be changed into a desired state. Also, a moving image can be displayed.

The display part 4 is fixed to a transport part 7 constructed by a belt etc. and the transport part 7 is wound on pulleys 8, 9. Since one pulley 8 is rotated and driven by a motor 10, the display part 4 linearly moves with rotation of the motor 10. In the present embodiment, rotational movement of the motor 10 is converted into linear movement by the pulley 8 and the transport part 7 of the belt etc., but the motor 10 and a feed screw may be combined or a linear motor may be used.

Also, the display part 4 is desirable to be arranged in a position moving so as to substantially dilate on an optical path of a sight line making connection between the eyepiece part 5 and the display part 4 with rotation of the motor 10 so that the trainee 2 can gaze at a character or a graphic form displayed on the display part 4 without moving up and down a sight line. In the embodiment, it is constructed so that the trainee 2 directly looks at the display part 4, but it may be constructed so as to miniaturize the apparatus by arranging a reflective device such as a mirror or a prism in the middle of the optical path and properly bending the optical path. Also, it can be constructed so that the whole is surrounded by the cabinet 3 to block the outside light in order to make the trainee 2 gaze at the display part 4.

Also, the motor 10, the shielding part 6 and the contents of display of the display part 4 are controlled by a main controller 11. An instruction signal or an operation from an input device 12 is inputted to this main controller 11 and also, the main controller 11 displays the contents of operation on an operation display part 13.

FIG. 2 is a block diagram specifically showing a configuration example of the main controller 11 in the first embodiment of the invention. The main controller 11 of this vision recovery training apparatus 1 is configured to include a central processing part 20 for controlling and managing an operation signal of each component, a display control part 21 for controlling the contents of display of the display part 4, a motor driving control part 22 for controlling driving of the motor 10, an opening and closing control part 23 for controlling opening and closing of the shielding part 6, an operation input part 24 for inputting an operation signal from the input device 12, an operation display control part 25 for controlling display of the operation display part 13, and storage device 26 for storing a training history. The central processing part 20 calculates a new training parameter based on the training parameter at least once applied to the trainee 2 and acquired from the storage device 26, and controls each part so as to conduct training to which the new training parameter calculated is applied. The display part 4 displays an image for vision training based on the new training parameter calculated by the central processing part 20. In addition, the storage device 26 may store the most suitable training parameter for the trainee 2 and changed every training. In accordance with this configuration, a new training parameter according to the newest state of the trainee can be calculated.

The display control part 21 controls display of background colors, colors, sizes, kinds, etc. of characters or graphic forms of the display part 4 based on a new training parameter calculated by the central processing part 20. The background colors, colors, sizes, kinds, etc. of these characters or graphic forms are training parameters unique to the trainee 2. The central processing part 20 has position information about the present display part 4 and can perform control so as to display a character or a graphic form of a size in proportion to an optical path length making connection between the eyepiece part 5 and the display part 4 through the display control part 21. Therefore, display size of an image for vision training can be changed so as to project the image for vision training on a retina of the trainee 2 in a constant size according to an optical distance between a position of the eye of the trainee 2 and a position of the display part 4 for displaying the image for vision training, and the trainee 2 can adjust an adjustment muscle so as to recover vision efficiently by gazing at the graphic form.

The motor driving control part 22 controls rotation driving of the motor 10 so that the display part 4 reciprocates between a near point 4a and a far point 4b by a predetermined number of times based on a new training parameter calculated by the central processing part 20. A movement speed profile for defining a speed etc. at which this display part 4 is reciprocated is instructed by the central processing part 20 of the main controller 11. A distance of the near point 4a, a distance of the far point 4b, the movement speed profile and the number of reciprocations are training parameters properly selected according to the extent, causes, etc. of a decrease in vision of the trainee 2. The motor driving control part 22 detects that the display part 4 is in any position, and notifies the central processing part 20.

The trainee 2 or a training instructor (or assistant) operates the input device 12, and inputs the training parameters, unique to the trainee 2, such as background colors, colors, sizes or kinds of the characters or graphic forms described above. Operation instructions accepted by the input device 12 are transmitted to the central processing part 20 through the operation input part 24, and the central processing part 20 gives control instructions necessary for each control block according to the contents of the operation instructions. The operation display part 13 displays parameter options, promotion, etc. of the operation instructions for the trainee 2 or the training instructor. This operation display part 13 is constructed by an electrical display device such as an organic EL display, a CRT display or a liquid crystal display. The training parameters, unique to the trainee 2, such as background colors, colors, sizes, kinds, etc. of characters or graphic forms displayed on the operation display part 13 are controlled by the operation display control part 25.

The storage device 26 stores a training history and the training parameters of each trainee 2. It is desirable to leave conducted training parameters and training conduct time in addition to the number of conducts of training as the training history. In addition, in the embodiment, the storage device 26 is configured to form a part of the main controller 11, but can also be transported by arranging the storage device outside the main controller 11 using a proper interface, and training parameters can also be shared between plural vision recovery training apparatus by connecting the storage device 26 through a network (not shown) and disposing an acquisition device acquires training histories such as the training parameters from the storage device 26 inside the vision recovery training apparatus. Also, a nonvolatile device such as a flash memory disk or a hard disk drive is suitable as the storage device 26.

The opening and closing control part 23 is connected to the shielding part 6 for opening and closing a window of the eyepiece part 5 in which the trainee 2 can peep with the eye, and controls opening and closing of the shielding part 6. The central processing part 20 sends an opening and closing control signal to the opening and closing control part 23 according to training methods for simultaneously conducting vision improvement training of both eyes of the trainee 2, or conducting the training of only one of both eyes, or conducting the training of one eye alternately. Instruction information about such training methods for simultaneously conducting vision improvement training of both eyes of the trainee 2, or conducting the training of only one of both eyes, or conducting the training of one eye alternately is also included in a training parameter.

Next, a training procedure using the vision recovery training apparatus 1 of the embodiment will be described. In the case of starting training, a trainee 2 or a training instructor operates predetermined buttons, keys, etc. of the input device 12 and thereby, inputs a sign or a mark such as a unique ID capable of specifying the trainee 2 or a name of the trainee 2 and notifies the central processing part 20 of a start of training. In that case, it may be constructed so that a microphone etc. are installed as the input device 12 and notification by sound can be provided.

In addition, when the trainee 2 is a first trainee, initial registration of the unique ID, the name, etc. of the trainee 2 is performed through the input device 12 and subsequently, initial registration of training parameters according to the extent, causes, etc. of a decrease in vision of the trainee 2 is performed.

The central processing part 20 refers to a sign or a mark unique to the trainee 2 notified, and acquires a training history of the trainee 2 from the storage device 26. In this case, training conduct time, information, etc. capable of distinguishing the number of conducts of training (the number of trainings) in addition to the training parameters conducted in the past are recorded as the training history. The central processing part 20 is programmed so that a training parameter suitable to be conducted this time is calculated from the training history such as the training parameters acquired from the storage device 26. When the trainee 2 is the first trainee, the training parameters in which the initial registration is performed are applied.

Here, in training for improving an image formation adjustment function of an eye by exercising a ciliary muscle, it is assumed that as the number of trainings is large and/or the training is periodically conducted at predetermined intervals, the image formation adjustment function is more improved and an accumulation effect of improvement is also high. Therefore, for a trainee in which the training is periodically conducted at predetermined intervals and the number of trainings is large, a training level is increased every training. That is, the image formation adjustment function improvement effect can be more increased by applying a training parameter with high difficulty. In reverse, for a trainee in which the training is irregularly conducted and the number of trainings is small, there is a possibility that the image formation adjustment function is not improved and when a level of training is increased more than necessary, fatigue is only caused and efficiency of training does not increase, so that it is unnecessary to increase the level of training.

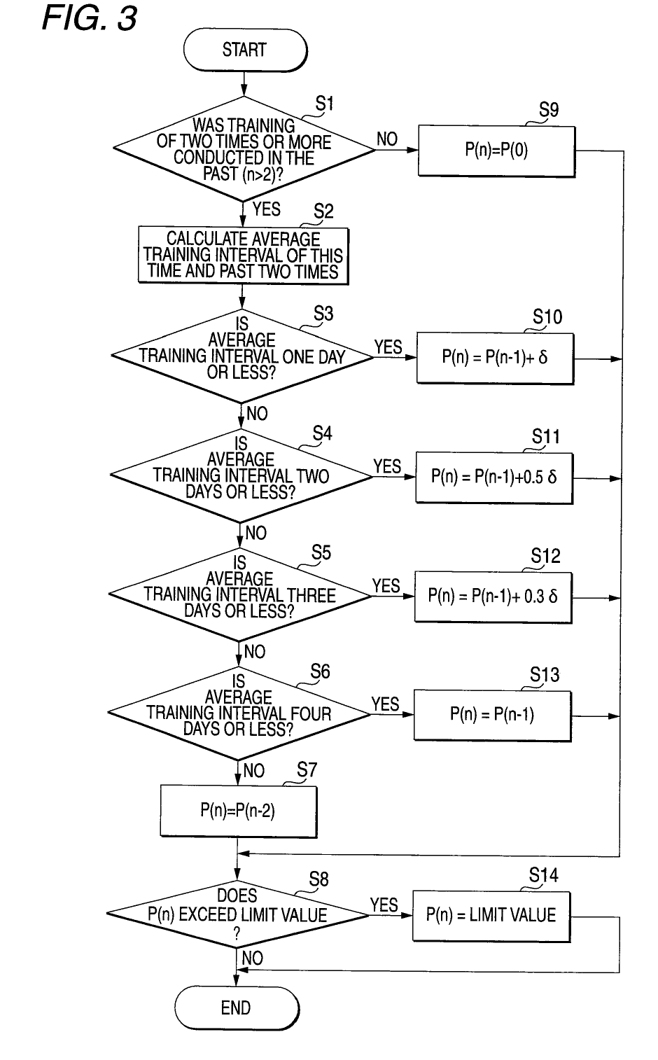

FIG. 3 is a flow diagram showing a calculation example of training parameters in the embodiment in consideration of the above. When the number of trainings is set at n and training of three times or more including the first time (n=0) and this time is not conducted (S1), training parameters registered at the first time are set at the training parameters of this time (S9) and when the training of three times or more is conducted (when the training of two times or more was conducted in the past (n>2)) (S1), an average number of days of the training interval is calculated (S2). Then, the amount of improvement of a training level is decided according to the average number of days of the training interval (S3 to S6), and training parameters obtained by adding the amount of improvement to the training parameters of the previous time are set at the training parameters of this time (S10 to S13). In the present example, the amount of improvement of a training level at the time when the average number of days of the training interval is one day is set at δ. The contents of δ are as follows.

Far point distance: +1 cm

Near point distance: −0.5 cm

Speed profile: +1 cm/s

Training time: +0.5 reciprocation

In the present example, 0.5δ, 0.3δ and 0δ are respectively added to the training parameters of the previous time every time the average number of days of the training interval extends by one day. In the case of exceeding four days, the training parameters of the previous time before last are applied (S7). Also, the training parameters have a limit value as image formation adjustment function training or a limit value on an apparatus configuration, so that when calculated training parameters reach this limit value, the training parameters are restricted so as not to exceed the limit value (S8, S14).

In addition, in the calculation example of the present training parameters, a training level is arithmetically improved with respect to the training parameters of the previous time, but can also be geometrically improved and the number of past trainings can also be given. Further, training parameters other than the four training parameters described above may be added.

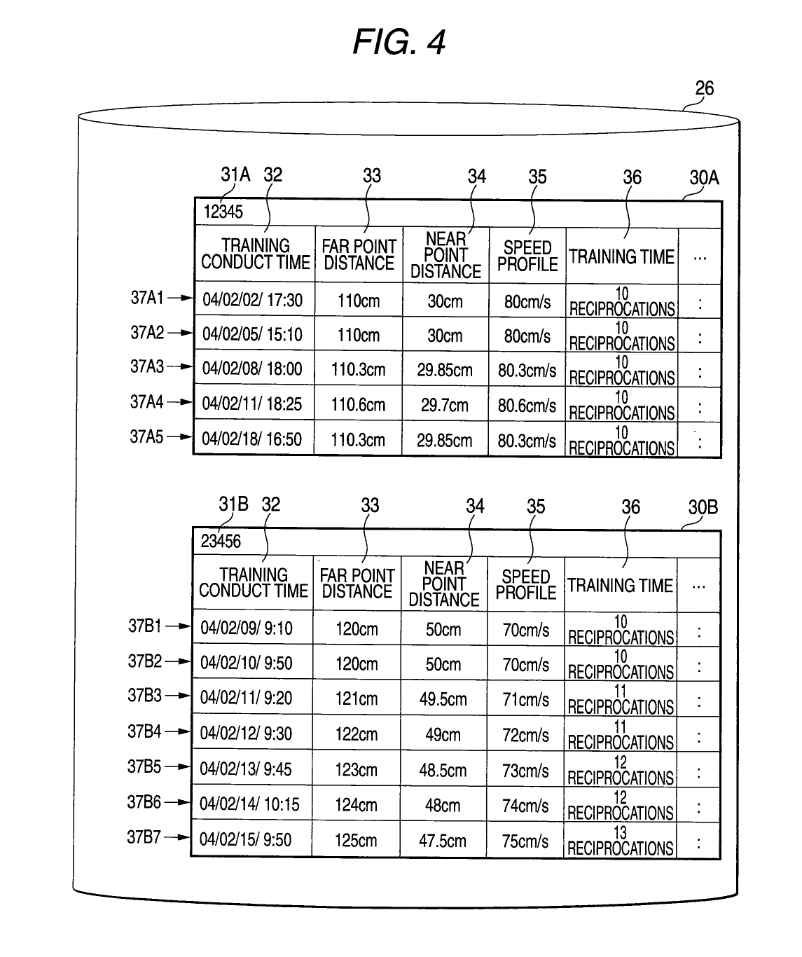

FIG. 4 is a diagram showing one example of a data format stored in the storage device 26 in the first embodiment of the invention, and herein shows data 30A, 30B of two trainees A, B. In the present example of the data format, one identification code 31A, 31B for specifying the trainee 2 and one or more training records used as a training history are provided. The central processing part 20 is programmed so as to add the training record every time training is conducted. This training record is made of training conduct time 32 and training parameters at that time, for example, a far point distance 33, a near point distance 34, a speed profile 35 and training time 36.

In the present example, the data 30A of the trainee A is constructed of the identification code 31A of the trainee A and five training records 37A1 to 37A5, so that it can be discerned that training was conducted five times in the past. The first training record 37A1 is training parameters in which initial registration is performed. The trainee A conducted training at intervals of three days from the first time to the fourth time and a training level was improved at the third time and the fourth time by a calculation flow of the training parameters programmed in the central processing part 20, but at the fifth time, the interval extended to seven days, so that the training parameters were returned to the training parameters of the third time.

On the other hand, the data 30B of the trainee B is constructed of the identification code 31B of the trainee B and seven training records 37B1 to 37B7. The trainee B conducted training every day from the first time to the seventh time and a training level was increased every training. Since a training interval is also shorter than that of the trainee A, the amount of improvement of a training level is also set larger than that of the trainee A.

According to the vision recovery training apparatus 1 of the embodiment thus, vision recovery training can be conducted efficiently by being constructed so that training can be conducted without inputting a training parameter according to an improvement state of an image formation adjustment function of the eye of each trainee 2 every training. Also, efficiency of training can be more improved by estimating the improvement state of the image formation adjustment function from a training history of each trainee and applying the most suitable training parameter to each trainee every training.

### Second Embodiment

The inventor found that there was a correlation between movement of a pupil (change in a diameter of the pupil) and a vision recovery effect (improvement state of an image formation adjustment function). A vision recovery training apparatus of a second embodiment is an apparatus constructed based on new knowledge of this inventor, and measures a diameter of the pupil of a trainee and conducts training to which a new training parameter decided based on a result of the measurement is applied.

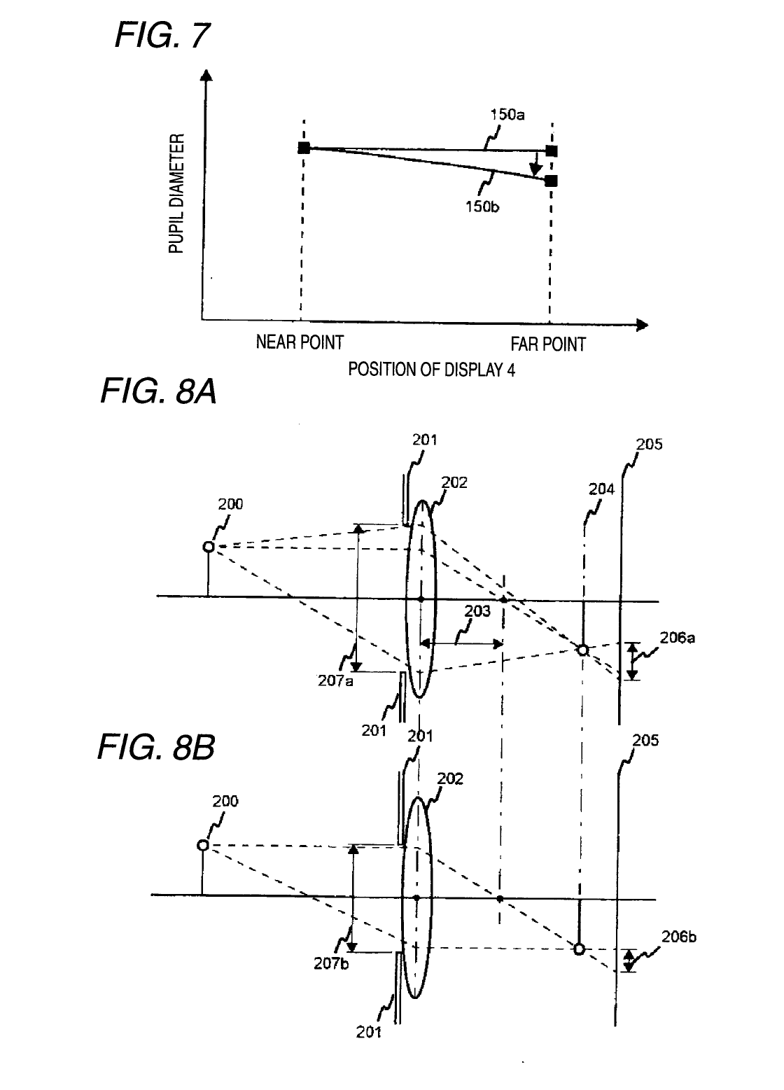

FIG. 7 is an example indicating how a change in a diameter of the pupil of a trainee 2 is made by increasing the number of vision recovery trainings. The axis of abscissa of FIG. 7 shows a position of a display part 4, that is, a visual target at which the trainee gazes, and this visual target reciprocates between a near point and a far point. The axis of ordinate of FIG. 7 shows a pupil diameter of the trainee. At the beginning of a start of vision recovery training, the pupil diameter is constant regardless of a position of the display part 4 as shown in a pupil diameter curve 150a. However, as the number of vision recovery trainings is increased, for example, for a myopic person, movement in which a pupil diameter contracts is shown when the display part 4 is in a far position as shown in a pupil diameter curve 150b. A change in movement of this pupil is probably an effect derived by an advantage of the vision recovery training apparatus of the invention in which training is conducted so that a size of a graphic form imaged on a retina becomes constant and a focal point of the eye continuously moves between a near point and a far point.

FIG. 8 is a schematic diagram of an index image formation state for describing action in which a visual target looks clearly when a pupil diameter contracts in a myopic person. FIG. 8(a) shows a state in which a diameter of a pupil 201 expands, and FIG. 8(b) shows a contraction state. Light passing through a visual target 200 passes through the pupil 201 and is refracted by a lens (crystalline lens) 202 of a focal distance 203 and converges on an image formation point 204. A retina 205 is located backward from the image formation point 204 by reasons that, for example, the focal distance 203 of the lens (crystalline lens) 202 of a myopic person is shorter than that of an emmetropic person or a distance from the lens (crystalline lens) 202 to the retina 205 of the myopic person is longer than that of the emmetropic person. Therefore, in the myopic person, a visual target image 206 on the retina of the visual target 200 is projected in a state diffused than the visual target image at the image formation point 204 and lightness reduces.

As a result of vision recovery training, for example, for a myopic person, a visual target image 206a is recognized by a pupil diameter 207a in the visual target 200 located far, but a visual target image 206b with reduced diffusion power can be recognized by a smaller pupil diameter 207b, so that lightness on the retina 205 improves, that is, vision improves.

From the above, a vision recovery effect can be measured by measuring a pupil diameter of the trainee 2 corresponding to a position of a visual target and calculating the amount of change in the movement.

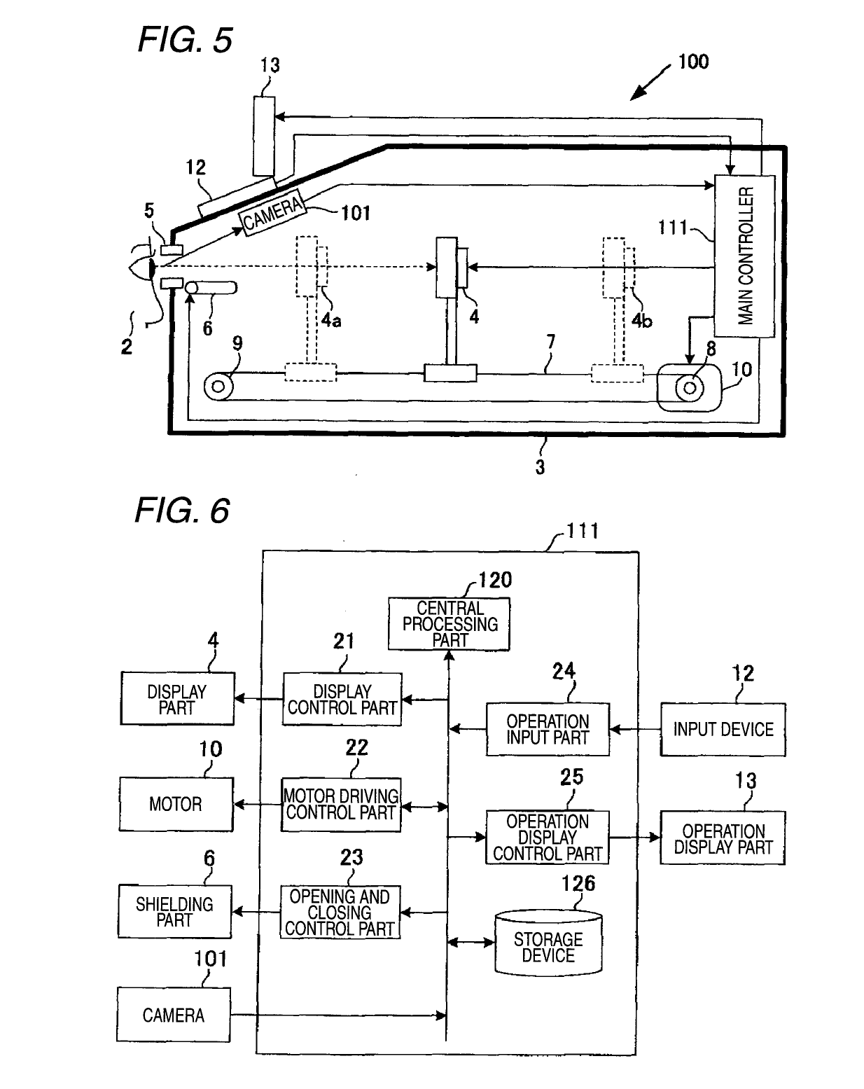

FIG. 5 is a diagram showing a configuration of the vision recovery training apparatus in the second embodiment of the invention, and FIG. 6 is a diagram specifically showing a configuration example of a main controller in the second embodiment of the invention. The vision recovery training apparatus 100 shown in FIG. 5 is an apparatus in which a camera 101 is added in the vision recovery training apparatus 1 shown in FIG. 1. Also, the vision recovery training apparatus 100 comprises a main controller 111 in which a function of receiving an input from the camera 101 is added to the function of the main controller 11 shown in FIGS. 1 and 2. In the configuration shown in FIGS. 5 and 6, the same numerals are assigned to the configuration described in the first embodiment and the description is omitted.

The camera 101 is an imaging device such as an infrared camera which captures an image of a pupil of a trainee, and sends the captured image to the main controller 111. A central processing part 120 of the main controller 111 measures a distance between the pupils and a diameter of the pupil of the trainee from the image received from the camera 101. As described above, a vision recovery training effect can be grasped from a result of the measurement. Also, the central processing part 120 refers to a table held by a storage device 126, and calculates a training parameter according to a change in the measured pupil diameter as a new training parameter, and controls each part so as to conduct training to which the calculated new training parameter is applied.

Table 1 shows one example of a table showing correspondence between training parameters and a change in a diameter of a pupil.

TABLE 1

| 34567 | (31C) | | | | | | (30C) | |
|---|---|---|---|---|---|---|---|---|
| TRAINING | | FAR POINT | NEAR POINT | | | | TRAINING | |
| CONDUCT | | PUPIL | PUPIL | FAR POINT | NEAR POINT | SPEED | TIME (RECIPRO- | |
| TIME(32) | | DIAMETER(38) | DIAMETER(39) | DISTANCE(33) | DISTANCE(34) | PROFILE(35) | CATIONS)(36) | . . . |
| 04/02/9/9:10 | | 6.6 mm | 6.6 mm | 120 cm | 50 cm | 70 cm/s | 10 | :(37C1) |
| 04/02/11/11:50 | | 6.3 mm | 6.6 mm | 120 cm | 50 cm | 70 cm/s | 10 | :(37C2) |
| . | | . | . | ↓ | ↓ | ↓ | ↓ | . |
| . | | . | . | | | | | . |
| . | | . | . | | | | | . |
| 04/03/15/10:20 | | 5.7 mm | 6.8 mm | 120 cm | 50 cm | 70 cm/s | 10 | :(37C3) |
| 04/03/18/9:30 | | 5.7 mm | 6.7 mm | 122 cm | 49 cm | 72 cm/s | 11 | :(37C4) |
| 04/03/20/1:45 | | 5.3 mm | 6.5 mm | 122 cm | 49 cm | 72 cm/s | 11 | :(37C5) |
| . | | . | . | ↓ | ↓ | ↓ | ↓ | . |
| . | | . | . | | | | | . |
| . | | . | . | | | | | . |
| 04/04/25/10:15 | | 4.5 mm | 6.8 mm | 122 cm | 49 cm | 72 cm/s | 11 | :(37C6) |
| 04/04/28/9:50 | | 4.6 mm | 6.5 mm | 124 cm | 48 cm | 74 cm/s | 12 | :(37C7) |

In Table 1, a far point pupil diameter 38 and a near point pupil diameter 39 are added to the example of a table format shown in FIG. 4. Here,

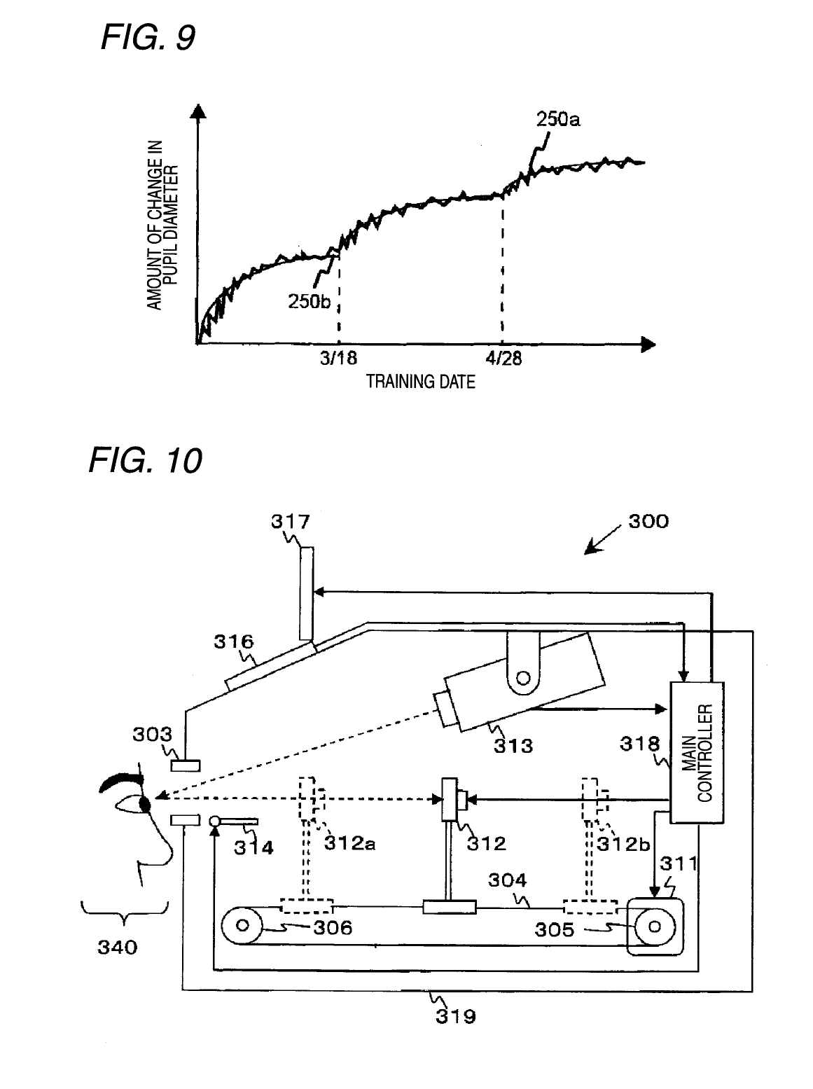

the amount of change in a pupil diameter=the far point pupil diameter 38−the near point pupil diameter 39
is used as a parameter for measuring a vision recovery training effect. Table 1 is a data example of a trainee C, and FIG. 9 shows the amount 250a of change in a pupil diameter with respect to a training date (calendar). According to this data, the amount of change in the pupil diameter increases smoothly from the beginning of training (February 9), but the amount of change in the pupil diameter does not increase on almost March 15. As a result of this, the central processing part 120 decides that a training effect decreases in the training parameters applied at that point in time, and training parameters for improving a training level are applied at a point in time of March 18. Similarly, the training level is improved on April 28.

Since the amount 250a of change in the pupil diameter has variations due to measurement environment, physical condition etc. of the trainee 2 actually, it is suitable to be offered to determination of a training effect by making conversion as shown in 250b by filter processing, function fitting processing, etc.

In addition, the present example refers to the parameter of (far point pupil diameter 38−near point pupil diameter 39) as the amount of change in the pupil diameter, but a rate of change in the pupil diameter (far point pupil diameter 38/near point pupil diameter 39) may be used. Further, a training effect determination method or the extent of improvement in a training level may be changed according to causes of a decrease in vision or the amount of change (rate of change) in the pupil diameter.

While it was conventionally necessary to measure a training effect using another apparatus etc. after training was conducted in order to grasp the training effect, according to the vision recovery training apparatus 100 of the embodiment, the training effect can be grasped during the training. Further, training can be conducted using a new training parameter according to a training effect properly without interrupting the training, so that efficient training can be conducted.

In addition, in the case of being constructed so as to block the outside light by forming a cabinet 3 for the purpose of, for example, improving a training effect, an infrared camera can also be used as the camera 101. In this case, it is suitable to use an infrared lamp (not shown) as illumination.

Further, in the case of arrangement in which the camera 101 cannot capture a pupil of the trainee 2 directly in the cabinet 3 for convenience of arrangement of each member constructing the invention, it may be constructed so as to take a photograph by adding a half mirror (not shown) on an optical path of a display part 4 and a viewpoint of the trainee 2 and further adding a mirror (not shown) as necessary.

### Third Embodiment

In the first and second embodiments, the vision recovery training apparatus capable of efficiently conducting vision recovery training and conducting training without inputting a training parameter according to an improvement state of an image formation adjustment function of each trainee every training has been described, but further in a third embodiment, a vision recovery training apparatus capable of applying the most suitable training parameter to each trainee without operating a parameter input etc. in the case of a start of training will be described.

FIG. 10 is a block diagram showing a configuration of a vision recovery training apparatus of the third embodiment of the invention. A configuration of a vision recovery training apparatus 300 of FIG. 10 shows a state in which a display part 312 is positioned in the middle of a far point 312b and a near point 312a. A trainee 340 of the present apparatus puts the eyes to an eyepiece part 303, and looks at a character or a graphic form displayed on the display part 312, and conducts vision improvement training. It is desirable that the eyepiece part 303 have a size in which a user can peep with both eyes, but one eyepiece part having a size of the extent capable of peeping with one eye is prepared and the user may conduct vision improvement training by one eye. Also, two eyepiece parts having a size of the extent capable of peeping with one eye may be prepared to simultaneously conduct vision improvement training of both eyes, or one eyepiece part 303 may be opened and closed by a shielding part 314 to conduct training of one eye or to alternately conduct training of right and left eyes. In addition, by disposing a lens in the eyepiece part 303 and forming a virtual image, the depth may be decreased to miniaturize the apparatus.

The display part 312 is a display device which displays an image for vision training, and is constructed by an electrical display device such as an organic EL (Electro-Luminescence) display, a CRT (Cathode Ray Tube) display or a liquid crystal display. Background colors, colors, sizes, kinds, etc. of characters or graphic forms displayed by the display part 312 can easily be changed into a desired state. Also, the display part 312 can display a moving image. Also, the display part 312 is fixed to a transport part 304 constructed by a belt etc. and the transport part 304 is wound on pulleys 305, 306. Since the pulley 305 is rotated and driven by a motor 311, the display part 312 linearly moves with rotation of the motor 311. In the present example, rotational movement of the motor 311 is converted into linear movement by the pulley 305 and the transport part 304 of the belt etc., but the motor 311 and a feed screw may be combined or a linear motor may be used.

The display part 312 is desirable to be arranged in a position moving so as to substantially dilate on an optical path of a sight line making connection between the eyepiece part 303 and the display part 312 with rotation of the motor 311 so that the trainee 340 can gaze at a character or a graphic form displayed on the display part 312 without moving up and down a sight line. In the present example, it is constructed so that the trainee 340 directly looks at the display part 312, but it may be constructed so as to miniaturize the apparatus by arranging a reflective device such as a mirror or a prism in the middle of the optical path and properly bending the optical path. Also, it is desirable to be constructed so that the whole vision recovery training apparatus 300 is surrounded by a cabinet 319 to block the outside light in order to make the trainee 340 gaze at the display part 312.

The vision recovery training apparatus 300 of the embodiment comprises a camera 313 for capturing a shape of an iris of an eyeball which is biological information about the trainee 340. The camera 313 is an acquisition device which acquires the biological information about the trainee 340, and when an iris of the trainee 340 is directly captured through the eyepiece part 303, an iris shape of the trainee 340 acquired is automatically encoded into an iris code by an encoder 323. The camera 313 which is an acquisition device has sensitivity outside a wavelength of visible light. Therefore, biological information having information about the outside of the wavelength of visible light can be acquired.

Biological information about an iris obtained by the camera 313 can be acquired in a state in which the trainee 340 conducts training. Further, a fingerprint sensor (not shown) for acquiring fingerprint information about a finger of the trainee 340 may be installed. Consequently, while the trainee 340 starts to use the vision recovery training apparatus 300 of the embodiment without any consciousness by only placing a fingertip on the fingerprint sensor, the vision recovery training apparatus 300 can automatically read the fingerprint optically in a training state and digitize data and authenticate personal information about the trainee 340 immediately. A sensor for sensing each information such as form information about a hand print, a palm print, an iris, a face, a blood vessel (vein), a retina, an ear print, etc., body information about voice, handwriting, etc., biochemical information about a blood type, DNA, etc. in addition to iris information and fingerprint information as information for personal authentication may be disposed. It may be constructed so that the vision recovery training apparatus 300 can automatically acquire each of the form information, the body information and the biochemical information by the sensor and digitize data and authenticate personal information about the trainee 340 immediately while the vision recovery training apparatus 300 starts to be used. It may be constructed so that personal information about the trainee 340 can be authenticated by properly selecting and combining these iris information, fingerprint information, form information, body information, biochemical information, etc. for personal authentication as necessary.

In the embodiment, the camera 313 directly captures an iris of the trainee 340 through the eyepiece part 303, but it goes without saying that the apparatus can be miniaturized by arranging a reflective device such as a mirror or a prism in the middle of an optical path making connection between the camera 313 and the eyepiece part 303 and properly bending the optical path. Also, when the whole apparatus is surrounded by the cabinet 319 in order to block the outside light, illumination by visible light cannot be used in view of its purpose, so that illumination (not shown) of the outside of a wavelength of visible light and a camera having sensitivity at a wavelength of the outside of visible light are used. For example, infrared illumination and an infrared camera are suitable.

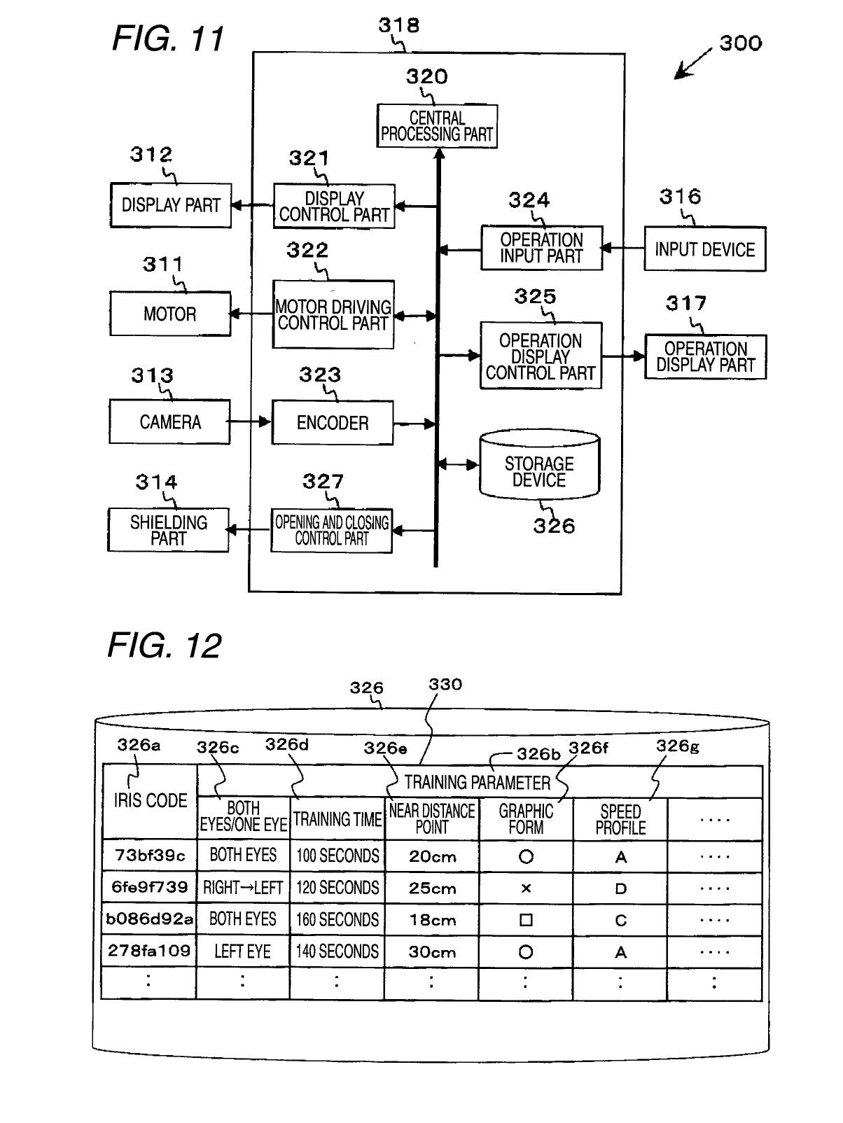

FIG. 11 is a block diagram specifically showing one example of a main controller of the vision recovery training apparatus of the third embodiment of the invention. A main controller 318 of the vision recovery training apparatus 300 includes a display control part 321, a motor driving control part 322, the encoder 323, an operation input part 324, an operation display control part 325, a storage device 326, and an opening and closing control part 327. A central processing part 320 controls and manages an operation signal of each component of the main controller 318. The display control part 321 is connected to the display part 312 for displaying graphic forms, characters, etc., and controls display of background colors, colors, sizes, kinds, etc. of the characters or the graphic forms displayed by the display part 312. The background colors, colors, sizes, kinds, etc. of the characters or graphic forms are training parameters unique to the trainee 340. The central processing part 320 has position information about the present display part 312, and the central processing part 320 can perform control so as to display a character or a graphic form of a size in proportion to an optical path length making connection between the eyepiece part 303 and the display part 312 using the display control part 321. Therefore, display size of an image for vision training is changed so as to project the image for vision training on a retina of a trainee in a constant size according to an optical distance between a position of the eye of the trainee and a position of the display part for displaying the image for vision training, so that the trainee 340 etc. can adjust an adjustment muscle so as to recover vision efficiently by gazing at the graphic form.

The motor driving control part 322 controls rotation driving of the motor 311 so that the display part 312 reciprocates between the near point 312a and the far point 312b by a predetermined number of times. A movement speed profile for defining a speed etc. at which the display part 312 is reciprocated is instructed by the central processing part 320 of the main controller 318. A distance of the near point 312a, a distance of the far point 312b, the movement speed profile and the number of reciprocations are training parameters properly selected according to the extent, causes, etc. of a decrease in vision of the trainee 340. Further, the motor driving control part 322 detects that the display part 312 is in any position, and notifies the central processing part 320.

The encoder 323 encodes an iris shape of the trainee 340 acquired by the camera 313 into an iris code. It is suitable for the encoder 323 to execute a program in which an encoding step is described by a processor. The processor may be included by the central processing part 320 or may be disposed for special use in the encoder 323. Also, a part of the encoding step is performed by a particular electronic circuit and the portion other than the part of the encoding step is performed by plural processors and distributed processing is performed and thereby, a processing speed and reliability improve.

The trainee 340 or a training instructor operates an input device 316 of the present apparatus, and inputs the training parameters, unique to the trainee 340, such as background colors, colors, sizes or kinds of the characters or graphic forms. Operation instructions accepted by the input device 316 are transmitted to the central processing part 320 through the operation input part 324, and the central processing part 320 gives control instructions necessary for each control block according to the contents of the operation instructions. An operation display part 317 displays parameter options, promotion, etc. of the operation instructions for the trainee or the training instructor. The operation display part 317 is constructed by an electrical display device such as an organic EL display, a CRT display or a liquid crystal display. The training parameters, unique to the trainee 340, such as background colors, colors, sizes, kinds, etc. of characters or graphic forms displayed on the operation display part 317 are controlled by the operation display control part 325.

The storage device 326 is a storage device which stores biological information about a trainee, and associates a series of inputted training parameters with an iris code and stores the training parameters in a database described below. The storage device 326 is connected through a network (not shown). By this configuration, authentication of biological information from a remote place through network communication can be implemented. A series of the past training parameters stored in the storage device 326 is retrieved as necessary by a retrieval device which retrieves biological information matched with the biological information acquired by the camera 313 which is an acquisition device, and a training parameter corresponding to the biological information retrieved by the retrieval device is selected by a selector and is extracted. The retrieval device and the selector are included in the central processing part 320.

The opening and closing control part 327 is connected to the shielding part 314 for opening and closing a window of the eyepiece part 303 in which a user can peep with the eye, and controls opening and closing of the shielding part 314. The central processing part 320 sends an opening and closing control signal to the opening and closing control part 327 according to training methods for simultaneously conducting vision improvement training of both eyes of the trainee 340, or conducting the training of only one of both eyes, or conducting the training of one of both eyes alternately. Instruction information about such training methods for simultaneously conducting vision improvement training of both eyes of the trainee 340, or conducting the training of only one of both eyes, or conducting the training of one of both eyes alternately is also a training parameter.

Next, a training procedure using the vision recovery training apparatus of the third embodiment of the invention will be shown. In the case of starting training, a trainee 340 operates predetermined buttons, keys, etc. of the input device 316 and thereby, notifies the central processing part 320 of the present apparatus of a start of training. In that case, it may be constructed so that a microphone etc. are installed as the input device 316 and start notification by sound can be provided, or it may be constructed so that a switch, a photo-interrupter, etc. are installed in the vicinity of the eyepiece part 303 and the trainee 340 peeps at the eyepiece part 303 and thereby a start of training is detected. Also, it is desirable to monitor the eyepiece part 303 by the camera 313 and detect a peep at the eyepiece part 303 of the trainee 340.

The central processing part 320 detecting the start of training converts a shape of an iris of the trainee 340 captured by the camera 313 into an iris code by the encoder 323. Next, the central processing part 320 retrieves an iris code with high matching among the past iris codes stored in the storage device 326 of the main controller 318. In this case, a threshold value of matching determination is properly selected so that a false reject rate which is an error in which the said person is not regarded as the said person and a false acceptance rate which is an error in which another person is regarded as the said person are set at a proper rate. In addition, a nonvolatile device such as a flash memory disk or a hard disk drive is suitable as the storage device 326.

FIG. 12 shows one example of a data format stored in the storage device in the embodiment. An iris code 326a is biological information respectively corresponding to each trainee 340. Training parameters 326b such as both eyes/one eye 326c, training time 326d, a near distance point 326e, a graphic form 326f, a speed profile 326g, . . . , etc. are stored in each trainee 340 corresponding to each iris code. In addition, the storage device may associate correspondence between the biological information and the training parameters with identification information (ID, name, etc.) about the trainee and store the correspondence. In this case, the training parameters can be extracted from a trainee ID or a trainee name. The retrieval device of the central processing part 320 retrieves the iris code 326a in a database 330 stored in the storage device 326. Then, the selector of the central processing part 320 selects and extracts the training parameters 326b corresponding to the iris code 326a authenticated as matching, and sets a necessary control parameter to each control part. Then, training is started.

Concretely, the trainee 340 having the iris code 326a of “73bf39c” trains over the training time 326d of “100 seconds” with eyes of “both eyes”, and conducts training based on the training parameters 326b such as the speed profile 326g, classified into, for example, “A” using the graphic form 326f of “O” shape at the near distance point 326e of “20 cm”. The trainee 340 having the iris code 326a of “6fe9f739” trains over the training time 326d of “120 seconds” with respective eyes in a direction “right→left” of the right eye to the left eye, and conducts training based on the training parameters 326b such as the speed profile 326g, . . . classified into, for example, “D” using the graphic form 326f of “x” shape at the near distance point 326e of “25 cm”. The trainee 340 having the iris code 326a of “b086d92a” trains over the training time 326d of “160 seconds” with eyes of “both eyes”, and conducts training based on the training parameters 326b such as the speed profile 326g, . . . classified into, for example, “C” using the graphic form 326f of “□” shape at the near distance point 326e of “18 cm”. The trainee 340 having the iris code 326a of “278fa109” trains over the training time 326d of “140 seconds” with the eye of “left eye”, and conducts training based on the training parameters 326b such as the speed profile 326g, . . . classified into, for example, “A” using the graphic form 326f of “O” shape at the near distance point 326e of “30 cm”.

When biological information matched with biological information acquired by the camera 313 which is an acquisition device is not present in the database 330 as a result of retrieval by the retrieval device of the central processing part 320, the vision recovery training apparatus 300 prompts the trainee 340 or a training instructor to input the training parameters, and the main controller 318 associates the inputted training parameters and the biological information acquired by the camera 313 which is an acquisition device with a trainee ID and stores the training parameters and the biological information in the storage device 326. Concretely, when an iris code of an iris of the trainee 340 converted by the encoder 323 cannot be detected as an iris code matched with the database 330 of the storage device 326, it is determined that the trainee 340 is a new trainee.

In this case, the central processing part 320 displays information such as graphic forms or characters for prompting an operator of the present apparatus to input the training parameters on the operation display part 317. The operator inputs the most suitable parameter for a vision recovery effect of the trainee 340 using the input device 316 according to parameter input instructions displayed on the operation display part 317. Also, the parameter input instructions may be given using sound by disposing a sound output device (not shown) such as a speaker, or may be displayed on the display part 312 when the contents of input instructions can be visually identified sufficiently. A series of the training parameters inputted in this manner are associated with the iris code 326a and are added to the database 330 of the storage device 326.

According to the vision recovery training apparatus 300 of the embodiment as described above, the trainee 340 only peeps at the eyepiece part 303 and thereby the trainee 340 can be pinpointed to select the training parameters for the trainee 340, so that training can be started with the training parameters suitable for each trainee without inputting the training parameters every training. Therefore, vision recovery training can be conducted efficiently.

The invention has been described in detail with reference to particular embodiments, but it is apparent to those skilled in the art that various changes or modifications can be made without departing from the spirit and scope of the invention.

The present application is based on Japanese Patent Application (No. 2005-196136) filed on Jul. 5, 2005, Japanese Patent Application (No. 2005-280054) filed on Sep. 27, 2005 and Japanese Patent Application (No. 2006-180964) filed on Jun. 30, 2006, the contents of which are incorporated herein by reference.

### INDUSTRIAL APPLICABILITY

The invention has an effect capable of efficiently conducting vision recovery training without inputting a training parameter every training since training according to an improvement state of an image formation adjustment function can be conducted for each trainee by conducting training to which a new training parameter based on a training parameter applied in the past is applied, and is useful for a vision recovery training apparatus etc. for conducting training of an image formation adjustment function of an eye.

### FIG. 1

11: MAIN CONTROLLER

### FIG. 2

4: DISPLAY PART 6: SHIELDING PART 10: MOTOR
12: INPUT DEVICE 13: OPERATION DISPLAY PART
20: CENTRAL PROCESSING PART 21: DISPLAY CONTROL PART
22: MOTOR DRIVING CONTROL PART
23: OPENING AND CLOSING CONTROL PART
24: OPERATION INPUT PART 25: OPERATION DISPLAY CONTROL PART
26: STORAGE DEVICE

### FIG. 3

START
S1: WAS TRAINING OF TWO TIMES OR MORE CONDUCTED IN THE PAST (n>2) ?
S2: CALCULATE AVERAGE TRAINING INTERVAL OF THIS TIME AND PAST TWO TIMES
S3: IS AVERAGE TRAINING INTERVAL ONE DAY OR LESS?
S4: IS AVERAGE TRAINING INTERVAL TWO DAYS OR LESS?
S5: IS AVERAGE TRAINING INTERVAL THREE DAYS OR LESS?
S6: IS AVERAGE TRAINING INTERVAL FOUR DAYS OR LESS?
S8: DOES P(n) EXCEED LIMIT VALUE?
S14: P(n)=LIMIT VALUE
END

### FIG. 4

32: TRAINING CONDUCT TIME
33: FAR POINT DISTANCE
34: NEAR POINT DISTANCE
35: SPEED PROFILE
36: TRAINING TIME
RECIPROCATIONS

### FIG. 5

101: CAMERA
111: MAIN CONTROLLER

### FIG. 6

4: DISPLAY PART
6: SHIELDING PART
10: MOTOR
12: INPUT DEVICE
13: OPERATION DISPLAY PART
21: DISPLAY CONTROL PART
22: MOTOR DRIVING CONTROL PART
23: OPENING AND CLOSING CONTROL PART
24: OPERATION INPUT PART
25: OPERATION DISPLAY CONTROL PART
101: CAMERA
120: CENTRAL PROCESSING PART
126: STORAGE DEVICE

### FIG. 7

PUPIL DIAMETER
NEAR POINT
FAR POINT
POSITION OF DISPLAY PART 4

### FIG. 9

AMOUNT OF CHANGE IN PUPIL DIAMETER
TRAINING DATE

### FIG. 10

318: MAIN CONTROLLER

### FIG. 11

311: MOTOR
312: DISPLAY PART
313: CAMERA
314: SHIELDING PART
316: INPUT DEVICE
317: OPERATION DISPLAY PART
320: CENTRAL PROCESSING PART
321: DISPLAY CONTROL PART
322: MOTOR DRIVING CONTROL PART
323: ENCODER
324: OPERATION INPUT PART
325: OPERATION DISPLAY CONTROL PART
326: STORAGE DEVICE
327: OPENING AND CLOSING CONTROL PART

### FIG. 12

326a IRIS CODE
326b TRAINING PARAMETER
326c BOTH EYES/ONE EYE
326d TRAINING TIME
326e NEAR DISTANCE POINT
326f GRAPHIC FORM
326g SPEED PROFILE
BOTH EYES
RIGHT→LEFT
LEFT EYE
SECONDS

1. A vision recovery training apparatus for training an image formation adjustment function of an eye, comprising:
a display device which displays an image for vision recovery training,
a training parameter calculator which calculates a new training parameter based on a training parameter which is acquired from a storage device which stores a training history and is applied to a trainee at least one time, and
a controller which conducts training to which the calculated new training parameter is applied,
wherein the controller performs at least one of control and display control of the display device and based on the new training parameter

2. (canceled)

3. (canceled)

4. A vision recovery training apparatus as claimed in claim 1, wherein the controller changes a display size of the image for vision training so as to project the image for vision training on a retina of the trainee in a constant size according to an optical distance between a position of an eye of the trainee and a position of the display device.

5. A vision recovery training apparatus as claimed in claim 1, wherein the controller performs movement control of the display device based on the new training parameter.

6. A vision recovery training apparatus as in of claim 1, wherein the storage device stores the number of past trainings of a trainee as the training history, and the training parameter calculator calculates the new training parameter based on the number of the past trainings.

7. A vision recovery training apparatus as in claim 1, wherein the storage device stores training conduct time as the training history, and the training parameter calculator calculates the new training parameter based on the training conduct time.

8. A vision recovery training apparatus as in of claim 1, wherein the storage device stores the new training parameter.

9. A vision recovery training apparatus as in claim 1, further comprising a diameter measurement device which measures a diameter of a pupil of a trainee, wherein the training parameter calculator refers to correspondence between a training parameter and a change in a diameter of a pupil measured and calculates a new training parameter.

10. A vision recovery training apparatus as claimed in claim 9, further comprising an imaging device which captures an image of a pupil of a trainee, wherein the diameter measurement device measures a diameter of a pupil of the trainee from an image captured by the imaging device.

11. A vision recovery training apparatus as in claim 1, further comprising an acquisition device which acquires biological information about a trainee, and a retrieval device which refers to a storage device which stores biological information about a trainee and retrieves biological information matched with the biological information acquired by the acquisition device.

12. A vision recovery training apparatus as claimed in claim 11, further comprising a selector which selects a training parameter corresponding to the biological information retrieved by the retrieval device.

13. A vision recovery training apparatus as claimed in claim 11, further comprising a controller in which when biological information matched with the biological information acquired by the acquisition device is not present as a result of retrieval by the retrieval device, an input of a training parameter is prompted and the inputted training parameter and the biological information acquired by the acquisition device are associated and are stored in the storage device.

14. A vision recovery training apparatus as in claim 11 to 13, wherein the biological information can be acquired in a state of conducting training.

15. A vision recovery training apparatus as claimed in claim 14, wherein the biological information is an iris of an eyeball.

16. A vision recovery training apparatus as claim 11, wherein the acquisition device acquires biological information by a camera having sensitivity outside a wavelength of visible light.

17. A vision recovery training apparatus as in claim 1, further comprising the storage device.

18. A vision recovery training apparatus as in claim 1, wherein the storage device is connected through a network.
